(19) United States
(12) Patent Application Publication
(10) Pub. No.: US 2017/0144124 A1
(43) Pub. Date: May 25, 2017

(54) Powdery Composition Containing Emulsion

(71) Applicant: Evonik Corporation, Parsippany, NJ (US)

(72) Inventors: Shabnam Navi Nejand, Hillsborough, NJ (US); Christopher R. Proulx, Piscataway, NJ (US)

(21) Appl. No.: 15/359,254

(22) Filed: Nov. 22, 2016

Related U.S. Application Data
(60) Provisional application No. 62/259,431, filed on Nov. 24, 2015.

(51) Int. Cl. B01J 13/20; B01J 13/08; A61K 8/25; A61K 8/06; A61Q 1/04; A61K 8/86; A61K 8/891; A61K 8/02; B01J 13/02; A61K 8/34

(52) U.S. Cl. CPC B01J 13/203; B01J 13/025; B01J 13/08; A61K 8/25; A61K 8/345; A61Q 1/04; A61K 8/86; A61K 8/891; A61K 8/0241; A61K 8/06; A61K 2800/60

(57) ABSTRACT

Powdery composition containing an emulsion having core-shell particles where the core is liquid and the shell is hydrophobized particles, and a carrier powder having a carrier and a partially water soluble liquid and/or a water reactive substrate reversibly bound to the carrier. A method of using the powdery composition is also disclosed whereby the powdery composition is applied and activated by energy input to form a creamy or liquid composition, and are suitable for various household and industrial applications including paint and stain products, surface treatment products, and personal care applications including cosmetic preparations, such as powders, in various products including, but not limited to, moisturizers, lotions, mascara, lipstick, lip gloss, sunscreen and fragrances. These free-flowing, fine dry water powdery compositions liquefy when applied, rubbed or brushed on the desired surface.

### CROSS REFERENCE TO RELATED APPLICATION

This U.S. patent application claims the benefit of and priority to U.S. Provisional Patent Application Ser. No. 62/259,431 filed Nov. 24, 2015, the entire disclosure of the application being considered part of the disclosure of this application, and hereby incorporated by reference.

### FIELD OF THE INVENTION

The invention relates to a powdery composition containing an emulsion and a process to produce a creamy composition from this powdery composition. Such powdery compositions are stable and may be stored in its powdery form until activated to the creamy composition by applying an energy input sufficient to release the water and dissolved or emulsified material of the emulsion from the powdery composition.

### BACKGROUND OF THE INVENTION

Solid cosmetic preparations, such as powders, having a high water content are known in the prior art. The basis of some patent applications is the long-known fact that in the presence of a hydrophobic powder, such as a hydrophobic silicon dioxide powder, water can be dispersed into fine droplets and enveloped by the hydrophobic material, thus preventing the droplets from rejoining. A powdery substance having a high water content, known as “dry water”, is formed in this way (Fine Particles series no. 11, Evonik Degussa). Cosmetic formulations based on “dry water” are described in Seifen, Fette, Öle, Wachse (SÖFW), 8 (2003), pages 1-8. These are free-flowing, fine powders, which liquefy when rubbed on the skin. Water-containing, liquefiable powder compositions based on this principle are further disclosed in several patent applications and patents.

U.S. Pat. No. 8,691,251 discloses a core shell particle comprising a shell and a core, the shell of which includes aggregated, hydrophobized silicon dioxide particles, and the core of which includes a liquid phase, and in which the ratio of aggregated, hydrophobized silicon dioxide particles to the liquid phase, based on the total weight of the particles, is 2:98 to 40:60, where 60-100% by weight of glycerol is present in the liquid phase.

U.S. Pat. No. 8,647,653 discloses a powdery composition comprising a core shell particle and carrier powder wherein the core shell particle contains a shell of hydrophobized silica particles and a core comprising water and up to 10 wt.-% of the total core weight of a dissolved or emulsified material, and the carrier powder contains precipitated silica as a carrier.

US 20070218024 discloses a powdered water cosmetic composition containing a water phase comprised of water droplets and a particulate phase comprised of non-wetting particulates which operate to surround and sequester the individual water droplets from each other in the composition.

US 20120021028 discloses a powder composition containing 50 to 90 wt. % of water, 0.5 to 10 wt. % of at least one other liquid having a boiling point higher than 150° C. at a pressure of 1013 mbar, at least one starch substituted with carboxy-(C₁ to C₄) alkyl groups, and metal oxide particles coated on the surface with fluorine-containing organic groups.

US 20140302106 discloses a powdery composition comprising core-shell particles having a shell of at least one hydrophobized metal oxide powder and a liquid core containing about 20 to 100% by weight of at least one polyol.

U.S. Pat. No. 4,274,883 discloses an aqueous dispersion of a hydrophobic silica consisting of 0.1 to 50 parts of hydrophobic silica, in a given case up to 3 parts of wetting agent and 99.9 to 50 parts of water. It is produced by intensive mixing of hydrophobic silica with water with the known “dry-water-phase” as an intermediate step. In a given case there can additionally be used a wetting agent in which case the “dry-water-phase” does not occur. The dispersion of hydrophobic silicas can be mixed without additional steps or materials with rubber latex.

DE-A-1467023 discloses the use of aqueous solutions comprising cosmetic ingredients instead of pure water for the preparation of dry water.

EP-A-1206928 discloses a water-containing powder composition comprising aqueous gel cores coated with hydrophobic particles.

JP 2000-309505 discloses a cosmetic powder being liquefied by embrocation including 2-20 wt. % of a hydrophobic silica having a specific surface area of at least 60 m2/g and an oil-in-water type emulsion composition.

EP-A-1235554 discloses a cosmetic or pharmaceutical powder-to-liquid composition comprising hydrophobically coated silica particles into which are incorporated water and a water soluble polymer, the composition containing less than 1% oil.

EP-A-1386599 discloses a method for producing dry water composed of an aqueous ingredient coated with a hydrophobic powder to form a powder state capable of liquefying upon embrocation at the time of use, comprising charging a hydrophobic powder and an aqueous ingredient into a hollow container forming a hydrophobic enclosed space in the inside thereof, followed by agitating at a high speed in the hydrophobic hollow container to form fine aqueous droplets, and then allowing the surface of the fine aqueous droplets to be uniformly adsorbed with the hydrophobic powder.

WO 01/85138 A2 discloses an encapsulation system comprising a core of aqueous liquid having at least 5% by weight water therein, and an encapsulant surrounding the core to form stable encapsulated particles, the encapsulant comprising at least one layer of hydrophobic particles in contact with and surrounding the core, the core and hydrophobic particles providing an encapsulated system that has a volume weighted mean particle diameter of from 0.05 to 25 micrometers, at least 25% of the encapsulated particles are spherical and can support its own weight.

EP-A-855177 discloses a whitening powder comprising 0.1 to 7 wt. % trimethylsiloxylated silica having a specific surface area of at least 80 m²/g and a hydrophobization degree of at least 50%, 5 to 40 wt. % of a polyhydric alcohol, 50 to 94 wt. % water and 0.01 to 5 wt. % of a whitening ingredient.

WO 2005/058256 discloses cosmetic preparations of creme- or paste-like consistency, comprising 50-95 wt. % water, hydrophobized silicon dioxide powder, at least one cosmetically-relevant active ingredient, or adjunct which is water soluble or which may be dispersed or emulsified in an aqueous medium and a viscosity regulator. The above is produced by continuously adding hydrophobized silicon dioxide powder with mixing, to a solution or a dispersion of at least one cosmetically-relevant active ingredient and the mixing is continued until a paste-like consistency is achieved.

Although prior art includes concepts such as the encapsulation of water, the problem with this concept is the amount of additives that could be added while maintaining a stable powder ranged from 0 to 3% by weight depending on additive properties such as the polarity and chain length of the additives. Limiting the formulation to 0 to 3% by weight of an additive, and limiting the additives, reduced the feasibility of the original concept. Even with guidance the industries that have tried using this concept had been unable to overcome the limitations regarding the variety of additives or additive amount.

### SUMMARY OF THE INVENTION

It is therefore an object of the present invention to provide a concept that overcomes past difficulties. The concept should not be restricted to cosmetic purposes, but should also allow introduction into other industries like personal care or household care and other industrial applications.

The present invention is directed to a powdery composition comprising a powdery composition, comprising: a) an emulsion comprising core-shell particles having a shell of hydrophobized particles and a core comprising: water and 10 to 95 wt.-% of a total core weight of a dissolved or emulsified material; and b) a carrier powder comprising a carrier and at least one of an at least partially water soluble liquid and/or a water reactive substrate reversibly bound to the carrier by at least one of capillary action, absorption and adsorption; the carrier comprises at least one material selected from the group consisting of silica, a silica-metal mixed oxide, fumed silica, phyllosilicate, starch, hollow glass spheres, nylon, sugar, cyclodextrines and polysaccharides, and wherein a weight ratio of the carrier powder to the core-shell particles is from 1:1 to 1:9, wherein the shell particles comprise hydrophobized silica particles, a BET surface area of the hydrophobized silica particles is from 200 to 290 m²/g, a carbon content of the hydrophobized silica particles is 2 to 4 wt.-%, a methanol wettability of the hydrophobized silica particles is at least 50, the hydrophobized silica particles are obtained by reacting a hydrophilic fumed silica having a BET surface area of from 270 to 330 m²/g with hexamethyldisilazane, wherein at least one of a) the core of the core-shell particles and b) the at least partially water soluble liquid and water reactive substrate comprises at least one material selected from the group consisting of a texturizer, preservative, pigment, exfoliant, UV light protection filter, an antioxidant, a moisturizing substance, a deodorant, an antiperspirant active compound, a biogenic substance, an insect repellent active compound, a bleaching agent, an antioxidant, a bleach, an antibacterial substance, an antifungal substance, a flavor, a fragrance, an acid, an alkali, and an enzyme.

In a preferred embodiment of the invention, the core of the powdery composition contains an amount of dissolved or emulsified material that increases or does not alter the surface tension of the core water. For this invention, the dissolved or emulsified materials that increase or does not alter the surface tension may be in the range from 10 wt.-% to 95 wt.-%, preferably from −40 wt.-% to −95 wt.-%, more preferably from 60 wt.-% to 90 wt.-%, based on the core. In some preferred embodiments, the dissolved or emulsified material is glycerine.

In other preferred embodiments according to the invention when the powdery composition contains at least the core shell particles and carrier powder, the dissolved or emulsified materials may be in the range from 30 wt.-% to 70 wt.-%, preferably from 40 wt.-% to 65 wt.-%, more preferably from 40 wt.-% to 50 wt.-%, more preferably from 55 wt.-% to 65 wt.-%, based on the total powdery composition.

It was unexpected the higher content of the dissolved or emulsified material (for example, glycerine), did not lower or altered the surface tension of the core water. This increased content of the dissolved or emulsified material provides an advantage over existing products. In this respect, the resulting powdery composition is more advantageous for personal care and cosmetic products that benefit from higher glycerine content such as, but not limited to, moisturizers, lotions, mascara, lipstick, lip gloss, sunscreen and fragrances.

The invention also contemplates a method of using the powdery composition such that the powdery composition is applied or delivered to the desired surface or object through various delivery means and devices prior to or after activation of the powdery composition by an energy input to a creamy or liquid composition. In instances when the powdery composition has been applied to the desire surface without activating the powdery composition, the powdery composition may then be activated by applying a suitable energy input, for example by shear force from applying finger pressure. In other instances, the powdery composition is activated as the the powdery composition is applied to the desired surface. In some instances, the powdery compositions are suitable for cosmetic preparations, such as powders, in various products including, but not limited to, moisturizers, lotions, mascara, lipstick, lip gloss, sunscreen and fragrances. In other instances, the powdery compositions are suitable for industrial and commercial applications including in paint and stain products and formulations, and other surface treatment products.

### DETAILED DESCRIPTION OF THE INVENTION

The water soluble liquid and the water reactive substrate are reversibly bound to the carrier by capillary action/absorption/adsorption and can be flushed or dissolved from the carrier, in part or completely, by the liquid water or the liquid aqueous phase of the core-shell particles. The carrier itself can be water soluble or not.

The core shell particles as part of the composition according to the invention refer to a powdered material, that contains liquid water or a liquid aqueous phase or an emulsion containing liquid water, but where the coalescence of the individual water droplets is prevented by a shell of hydrophobic or hydrophobized particles. The shell is able to release the liquid aqueous phase or liquid water from the core when activated by pressure, shear, temperature, vibration and/or addition of one or more solvents or one or more surfactants. These kind of core shell particles are known as “dry water”. The aqueous phase may be an aqueous solution, an aqueous dispersion or an aqueous emulsion.

It has been unexpectedly found that an aqueous emulsion containing up to 95 wt.-% of the total core shell particle of a dissolved or emulsified material maintained the “dry water” characteristics such that the aqueous emulsion dispersed into fine droplets and enveloped by the hydrophobic or hydrophobized particles. To achieve the dry water characteristics, the dissolved or emulsified material should neither significantly reduce the surface tension of the core nor cause wetting of the hydrophobic or hydrophobized particles of the shell.

Hydrophobic particles are understood to be hydrophobic particles per se, whereas hydrophobized particles are those obtained by reacting the surface of a hydrophilic particle with a hydrophobic surface modifying agent. Usually a covalent bond is formed between reactive groups at the surface of the particles, e.g. hydroxyl groups, and reactive groups of the surface modifying agent.

In general “hydrophobic” means that there is only a small or no interaction with water and the affinity with water is small or zero. A methanol wettability may be used as a quantitative measure of the hydrophobicity. The shell particles of the present invention should have a methanol wettability of at least 40, preferably 50 to 80, more preferably 60 to 70. For the determination of the methanol wettability, in each case 0.2 g (±0.005 g) of hydrophobic or hydrophobized particles are weighed into transparent centrifuge tubes. 8.0 ml portions of a methanol/water mixture with 10, 20, 30, 40, 50, 60, 70 or 80 vol. % methanol are added to each sample. The tubes are shaken for 30 seconds and then centrifuged at 2500 min⁻¹ for 5 minutes. The methanol wettability is the defined as the volume percent of methanol for which the sediment volume is 100%. The higher the number the higher the hydrophobicity.

The hydrophobic or hydrophobized shell of the core shell particles usable in the present invention comprise for example inorganic powders such as talc, kaolin, mica, sericite, dolomite, phlogopite, synthetic mica, lepidolite, biotite, lithia mica, vermiculite, magnesium carbonate, calcium carbonate, aluminum silicate, barium silicate, calcium silicate, magnesium silicate, strontium silicate, tungstenic acid metal salts, magnesium, silica, zeolite, barium sulfate, calcined calcium sulfate, calcium phosphate, fluorapatite, hydroxyapatite, titania, fumed titania, alumina and fumed alumina.

Among these hydrophobized silica particles which form a three dimensional network, an aggregated structure, are a preferred shell material. The silica may be a precipitated silica or a fumed silica, the latter being preferred. Fumed silica is obtained in a flame hydrolysis or flame oxidation process. Its purity is higher than 99 wt.-%, usually higher than 99.8 wt.-%. Fumed silica usually forms a three-dimensional network of aggregated primary particles. The fumed silica primary particles bear hydroxyl groups at their surface and are nonporous.

Precipitated and fumed silica particles, as well as other particles suitable as shell particles are hydrophilic particles which need to be hydrophobized in a subsequent step. Procedures for this step are known for the person skilled in the art.

Suitable surface modifying agents can be the following silanes, which may be used individually or as a mixture.

Organosilanes (RO)₃Si(CₙH₂ₙ₊₁) and (RO)₃Si(CₙH₂ₙ₋₁) with R=alkyl, such as methyl, ethyl, n-propyl, isopropyl, butyl and n=1-20.

Organosilanes R′ₓ(RO)_{y}Si(CₙH₂ₙ₊₁) and R′ₓ(RO)_{y}Si(CₙH₂ₙ₋₁) with R=alkyl, such as methyl, ethyl, n-propyl, isopropyl, butyl; R′=alkyl, such as methyl, ethyl, n-propyl, isopropyl, butyl; R′=cycloalkyl; n=1-20; x+y=3, x=1, 2; y=1, 2.

Haloorganosilanes X₃Si(CₙH₂ₙ₊₁) and X₃Si(CₙH₂ₙ₋₁) with X=Cl, Br; n=1-20. Haloorganosilanes X₂(R′)Si(CₙH₂ₙ₊₁) and X₂(R′)Si(CₙH₂ₙ₋₁) with X=Cl, Br, R′=alkyl, such as methyl, ethyl, n-propyl, isopropyl, butyl-; R′=cycloalkyl; n=1-20.

Haloorganosilanes X(R′)₂Si(CₙH₂ₙ₊₁) and X(R′)₂Si(CₙH₂ₙ₋₁) with X=Cl, Br; R′=alkyl, such as methyl-, ethyl-, n-propyl-, isopropyl-, butyl-; R′=cycloalkyl; n=1-20.

Organosilanes (RO)₃Si(CH₂)ₘ—R′ with R=alkyl, such as methyl-, ethyl-, propyl-; m=0-20, R′=methyl, aryl such as —C₆H₅, substituted phenyl radicals, C₄F₉, OCF₂—CHF—CF₃, C₆F₁₃, OCF₂CHF₂, Sₓ—(CH₂)₃Si(OR)₃.

Organosilanes (R″)ₓ(RO)_{y}Si(CH₂)ₘ—R′ with R″=alkyl, x+y=3; cycloalkyl, x=1, 2, y=1, 2; m=0.1-20; R′=methyl, aryl, such as C₆H₅, substituted phenyl radicals, C₄F₉, OCF₂—CHF—CF₃, C₆F₁₃, OCF₂CHF₂, Sₓ—(CH₂)₃Si(OR)₃, SH, NR′R″R′″ with R′=alkyl, aryl; R″=H, alkyl, aryl; R′″=H, alkyl, aryl, benzyl, C₂H₄NR″″R with R″″=H, alkyl and R′″″=H, alkyl.

Haloorganosilanes X₃Si(CH₂)ₘ—R′ with X=Cl, Br; m=0-20; R′=methyl, aryl such as C₆H₅, substituted phenyl radicals, C₄F₉, OCF₂—CHF—CF₃, C₆F₁₃, O—CF₂—CHF₂, Sₓ—(CH₂)₃Si(OR)₃, where R=methyl, ethyl, propyl, butyl and x=1 or 2, SH.

Haloorganosilanes RX₂Si(CH₂)ₘR′ with X=Cl, Br; m=0-20; R′=methyl, aryl such as C₆H₅, substituted phenyl radicals, C₄F₉, OCF₂—CHF—CF₃, C₆F₁₃, O—CF₂—CHF₂, —OOC(CH₃)C═CH₂, —Sₓ—(CH₂)₃Si(OR)₃, where R=methyl, ethyl, propyl, butyl and x=₁ or 2, SH.

Haloorganosilanes R₂XSi(CH₂)ₘR′ with X=Cl, Br; m=0-20; R′=methyl, aryl such as C₆H₅, substituted phenyl radicals, C₄F₉, OCF₂—CHF—CF₃, C₆F₁₃, O—CF₂—CHF₂, —Sₓ—(CH₂)₃Si(OR)₃, where R=methyl, ethyl, propyl, butyl and x=1 or 2, SH.

Silazanes R′R₂SiNHSiR₂R′ with R,R′=alkyl, vinyl, aryl.

Cyclic polysiloxanes D3, D4, D5 and their homologs, with D3, D4 and D5 meaning cyclic polysiloxanes having 3, 4 or 5 units of the type —O—Si(CH₃)₂, e.g. octamethylcyclotetrasiloxane=D4.

Polysiloxanes or silicone oils of the type Y—O[[—SiRR′—O]ₘ—[SiR″R′″—O]ₙ]ᵤ—Y with R=alkyl; R′=alkyl, aryl, H; R″=alkyl, aryl; R′″=alkyl, aryl, H; Y=CH₃, H, C_{z}H_{2z+1} with z=1-20, Si(CH₃)₃, Si(CH₃)₂H, Si(CH₃)₂OH, Si(CH₃)₂(OCH₃), Si(CH₃)₂(C_{z}H_{2z+1}) and z=1-20, m=0, 1, 2, 3, . . . , n=0, 1, 2, 3, . . . , u=0, 1, 2, 3, . . . .

As surface modifying agents the following compounds may be preferred: octyltrimethoxysilane, octyltriethoxysilane, hexamethyldisilazane, hexadecyltrimethoxysilane, hexadecyltriethoxysilane, dimethylpolysiloxane, nonafluorohexyltrimethoxysilane, tridecafluorooctyltrimethoxysilane, tridecafluorooctyltriethoxysilane. With particular preference it is possible to use hexamethyldisilazane, octyltriethoxysilane and dimethyl polysiloxanes.

The shell particles of the powdery composition according to the invention can be hydrophobized silica particles having a BET surface area preferably from 30 m²/g to 500 m²/g, more preferably of 100 m²/g to 350 m²/g. Due to the reaction with the surface modifying agent these particles may contain 0.1 to 15 wt.-%, usually 0.5. to 5 wt.-%, of carbon.

Typical examples that may be used as shell material are AEROSIL® R104 (octamethylcyclotetrasiloxane; 150 m²/g; 55); AEROSIL® R106 (octamethylcyclotetrasiloxane; 250 m²/g; 50), AEROSIL® R202 (polydimethylsiloxane; 100 m²/g; 75), AEROSIL® R805 (octylsilane; 150 m²/g; 60), AEROSIL® R812 (hexamethyldisilazane; 260 m²/g; 60), AEROSIL® R812S (hexamethyldisilazane; 220 m²/g; 65), AEROSIL® R8200 (hexamethyldisilazane; 150 m²/g; 65), HDK® H2000, HDK® H2050, and HDK® H3004 from Wacker, Cab-O-Sil® brand such as Cab-O-Sil® TS-530 which is silica treated with hexamethyldisilazane, Cab-O-Sil® TS 720 which is silica treated with dimethicone from Cabot Corporation.

The indication in parenthesis refer to the surface modifying agent, the approximate BET surface area and the approximate methanol wettability, determined by the procedure given in this specification.

Particularly useful shell particles material are hydrophobized silica particles which are obtained by reacting a hydrophilic fumed silica particles having a BET surface area of from 270 to 330 m²/g with hexamethyldisilazane. The obtained hydrophobized silica particles have a BET surface area of from 200 to 290 m²/g, a carbon content of 2 to 4 wt.-% and a methanol wettability of at least 50, more preferred 50 to 70.

It may also be beneficial to use hydrophobized fumed silica particles in compacted form or as granules.

The other part of the core shell particles to be discussed is the core. The core contains liquid water, either in form of liquid water itself, or as a liquid aqueous solution, an emulsion containing liquid water or as an aqueous dispersion. The dissolved or emulsified material in case of a liquid aqueous solution or emulsion or the solid material in case of an aqueous dispersion should be selected from those materials that neither significantly reduce the surface tension of the core nor cause wetting of the hydrophobic or hydrophobized particles of the shell. Furthermore, in some embodiments, a dissolved or emulsified material that increases or does not alter the surface tension of the water is preferred. For the liquid aqueous solution or emulsion this means that the dissolved or emulsified materials that increase or does not alter the surface tension may be in the range from 10 wt.-% to 95 wt.-%, preferably from −40 wt.-% to −95 wt.-%, more preferably from 60 wt.-% to 90 wt.-%, based on the core. In some embodiments, the solution contains one of 58, 59, 60, 61, 62, 63, 64, 65, 66, 84, 85, 86, 87, 88, 89, 90, 91, 92, 93, 94 or 95 wt.-% of the dissolved or emulsified material of the solution, based on the core.

In some embodiments according to the invention when the powdery composition contains at least the core shell particles and carrier powder, the dissolved or emulsified materials may be in the range from 30 wt.-% to 70 wt.-%, preferably from 40 wt.-% to 65 wt.-%, more preferably from 40 wt.-% to 50 wt.-%, more preferably from 55 wt.-% to 65 wt.-%, based on the total powdery composition. In special embodiments of powdery compositions that are used in cosmetic applications, for example Powder-to-Cream Matte Lip Product Formulations, the dissolved or emulsified material may be 40, 41, 42, 43, 44, 45, 46 or 47 wt.-% of the total powdery composition. In special embodiments of powdery compositions that are used in cosmetic applications, for example Powder-to-Cream Glossy Lip Product Formulations, the dissolved or emulsified material may be 58, 59, 60, 61, 62 or 63 wt.-% of the total powdery composition.

The core can consist of a liquid aqueous phase containing up to 75 wt.-% of compounds typically used in the fields of cosmetic, personal care or household care may be used, as long as they do not reduce the surface tension of the core nor cause wetting of the hydrophobic or hydrophobized particles of the shell. Typical example will be discussed together with the carrier containing a at least partially water soluble liquid and/or a water reactive substrate.

The core content of the core-shell particles is usually in a range from 75 to 99 wt.-%, more preferably 85 to 95 wt.-%, based on the core shell particle. In some embodiments, the core content of the core-shell particles is 87, 88, 89, 90, 91, 92, 93, 94, or 95 wt.-% based on the core shell particle.

The dissolved or emulsified material of the core includes at least one material selected from ethylene glycol, butylene glycol, 2,3-butandiol, propylene glycol, dipropylene glycol, tripropylene glycol, hexylene glycol, glycerine, diglycerine, glucose, fructose, lactose, saccharose, maltose, mannitol, mannose, PEG-4 to PEG-800, like PEG-4, PEG-6, PEG-7, PEG-8, PEG-9, PEG-10, PEG-12, PEG-14, PEG-16, PEG-18, PEG-20, sorbitol, polyglyceryl sorbitol, urea, xylitol and mixtures thereof.

The powdery composition according to the invention consists of a powder next to the core shell particles that consists of at least one other powder, defined as a carrier powder containing a carrier and at least one of an at least partially water soluble liquid or a water reactive substrate or an emollient mixture, each located in and/or on the carrier.

The carrier is preferably selected from the group consisting of silica, fumed silica, silica-metal mixed oxides, like silica-alumina mixed oxides, silica-titania mixed oxides, phyllosilicate, clay, diatomaceous earth, starch, hollow glass spheres, nylon, sugar, cyclodextrines and polysaccharides.

In a special embodiment of the invention the carrier is selected from fumed silica or precipitated silica, good results being obtained with hydrophilic as well as hydrophobized forms.

In another special embodiment of the invention the carrier is a precipitated silica having

a) a BET surface area from 50 to 1000 m²/g, preferably from 100 to 700 m²/g, more preferably 150 to 500 m²/g,

b) a particle size d50, determined by laser diffraction, from 2 to 600 μm, preferably from 4 to 600 μm, preferably from 2 to 400 μm, preferable from 4 to 50 μm, preferably from 3 to 20 μm, more preferably from 4 to 10 μm, and

c) a DBP (dibutyl phthalate) absorption, in g DBP/100 g silica, of from 200 to 400, preferably 250 to 350.

The BET surface area is determined according to ISO 5794-1, annex D, the particle size d50 is determined according to ASTM 690-1992, and the DBP absorption is determined according to ASTM D 2414, based on the substance after drying.

Special grades of precipitated silica comprise a material having a BET surface area of from 400 to 500 m²/g, a particle size d50 of 4 to 6 μm and a DBP absorption of 300 to 350 g DBP/100 g silica. Also another material having a BET surface area of from 150 to 250 m²/g, a particle size d50 of 4 to 7 μm and a DBP absorption of 250 to 300 g DBP/100 g silica. Typical examples are SIPERNAT® 500 LS and SIPERNAT® 22 LS, both from Evonik Corporation.

In one embodiment, the precipitated silica comprise a material having a BET surface area of 475 m²/g, a particle size d50 of 6 μm and a DBP absorption of 325 g DBP/100 g silica.

In another special embodiment of the invention the carrier is a fumed silica having a BET surface area from 100 to 350 m²/g, preferably from 200-300 m²/g.

The water soluble liquid is capable of being flushed or dissolved from the carrier, in part or completely, by the core material of the core-shell particles in case the core shell particles are activated by pressure, shear, temperature, vibration and/or addition of one or more solvents or one or more surfactants. This means that the water soluble liquid is either partially or completely dissolved from carrier by the water or the aqueous phase of the core. Or the water soluble liquid can be flushed, partially or completely, form carrier by the water or the aqueous phase of the core.

The same applies for a water reactive substrate. A water reactive substrate is a material which is not water soluble or soluble only to a small amount. When being activated by water or an aqueous phase it is transformed to a reaction product or products that are essentially or completely water soluble.

In some instances, the water reactive substrate may contain an emollient mixture which is a material or mixture of materials which are not water soluble. The emollient mixture provides at least one of a humectant, lubricant and an occluder. Occlusion provides a layer of oil on the skin's surface, thus slowing down water loss. A humectant enhances the surface of the skin's capacity to hold water. A lubricant reduces friction when anything rubs against the skin.

The emollient mixture is preferably selected from one or more of Argon Oil, Butylene Glycol, C12-C15 Alkyl Benzoate, Caprylic/Capric Triglyceride, Castor, Cyclopentasiloxane, Decamethylcyclopentasiloxane, Ethylhexyl Palmitate/Tegosoft OP, Glycerin, Grapeseed, Hexadecanoic Acid, 2-ethylhexyl ester Isocetyl Palmitate, Jojoba, Macadamia, Mineral Oil, Olive, Paraffin Oil, PEG 400, Polydimethylsiloxane, PPG-15 Stearyl/Ether, Propylene Glycol, Rapeseed, Silicone 350, Silicone Fluid 50, Soybean, Soybean Oil, and Sunflower Oil.

The weight ratio of the at least partially water soluble liquid and/or water reactive substrate and/or emollient mixture to carrier is determined by multiple factors such as the viscosity of the water soluble liquid, the type of emollient mixture used, the structure of carrier comprising the pore size, the pore volume and the BET surface area. Usually is in the range of 10 to 90, a range from 60 to 75 being preferred.

The weight ratio of the carrier including the at least partially water soluble liquid and/or water reactive substrate and/or emollient mixture to the core-shell particles usually is from 1:1 to 1:9.

The average particle size of the carrier and of the core-shell particles, determined for example by transmission electron microscopy, usually can vary, independently from each other, from 2 to 20 μm.

In some embodiments, a free-flow agent (anticaking agent) may be added to the powdery composition. Free flow agents function to prevent lumping of the carrier and core shell particles of the powdery composition. Examples of free flow agents include, but are not limited to, silica, fumed silica, silica-metal mixed oxides, like silica-alumina mixed oxides, silica-titania mixed oxides, phyllosilicate, clay, diatomaceous earth, starch, hollow glass spheres, nylon, sugar, cyclodextrines and polysaccharides. In some instances, the free-flow agent may be identical in material to the shell particles of the core shell particles. In one special embodiment, the free-flow agent is AEROSIL® R812S (Evonik Corporation).

The aqueous phase of the core of the core-shell particles and/or the at least partially water soluble liquid and/or water reactive substrate, each in and/or on the carrier comprises at least one of the materials of the group consisting of comprises at least one of the materials of the group consisting of texturizers, preservatives, pigments, exfoliant, UV light protection filters, antioxidants, moisturizing substances, deodorant, antiperspirant active compounds, biogenic substances, insect repellent active compounds, bleaching agents antioxidant, bleach, antibacterial substances, antifungal substances, flavors, fragrances, acids, alkalis, enzymes or mixtures thereof.

Texturizers according to the invention, are materials that provide texture to the powdery composition such as a smooth and/or soft feel, and can thicken or add slip to a product, so it can be applied smoothly. Examples of suitable texturizers can be carbomer, polymers, inorganic powders such as talc, kaolin, mica, sericite, dolomite, phlogopite, synthetic mica, lepidolite, biotite, lithia mica, vermiculite, magnesium carbonate, calcium carbonate, aluminum silicate, barium silicate, calcium silicate, magnesium silicate, strontium silicate, tungstenic acid metal salts, magnesium, silica, zeolite, barium sulfate, calcined calcium sulfate, calcium phosphate, fluorapatite, hydroxyapatite, titania, fumed titania, alumina and fumed alumina. In one embodiment, the texturizer is SHPERON N-2000J® (Presperse Corporation).

Preservatives according to the invention, are natural or synthetic materials that inhibit microbial growth or undesirable chemical changes to prevent spoilage of products such as foods, pharmaceuticals and personal care formulations including powdery compositions. Examples of suitable preservatives can be phenoxyethanol, methylparaben, ethylparaben, butylparaben, propylparaben, isobutylparaben, benzoic acid, benzyl alcohol, benzylhemiformal, benzylparaben, 5-bromo-5-nitro-1,3-dioxane, 2-bromo-2-nitropropane-1,3-diol, butyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference, and mixtures thereof. In one embodiment, the preservative is PHENONIP® (Clariant International Ltd.).

Exfoliants according to the invention slough away the dead skin cells that have accumulated on the surface of your skin in order to maintain youthful, healthy-looking skin that glows and remains free of blemishes. Exfoliants are also used in keeping the skin free of wrinkles. There are three main types of exfoliation: mechanical, chemical and enzyme. In the case of this invention, we can deliver chemical or enzymatic exfoliants to the skin. Examples of suitable exfoliants include, but are not limited to, precipitated Silica, Sodium tetraborate decahydrate granules, Aluminum oxide, Ground seeds, such as apricot, almond and walnut seeds, Calcium carbonate, Polyethylene silica or beads, and Alpha Hydroxy Acids. In some embodiments, the exfoliant is precipitated silica having a size range from 50 to 600 μm, preferably from 110 to 320 μm. In some embodiments, the exfoliant is incorporated in the carrier powder.

UV light protection filters, according to the invention, are organic substances which are liquid or crystalline at room temperature and are capable of absorbing ultraviolet rays and of releasing the absorbed energy again in the form of radiation of longer wavelength, for example heat. UV filters can be oil-soluble or water-soluble. Oil-soluble substances are for example:
3-benzylidenecamphor and 3-benzylidenenorcamphor and derivatives thereof,
4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester- esters of cinnamic acid, preferably 4-methoxycinnamic acid 2-ethylhexyl ester, 4-methoxycinnamic acid propyl ester, 4-methoxycinnamic acid isoamyl ester, 2-cyano-3,3-phenylcinnamic acid 2-ethylhexyl ester (octocrylene)
esters of salicylic acid, preferably salicylic acid 2-ethylhexyl ester, salicylic acid 4-isopropylbenzyl ester, salicylic acid homomenthyl ester
derivatives of benzophenone, preferably 2-hydroxy-4-methoxybenzophenone, 2-hydroxy-4-methoxy-4′-methylbenzophenone, 2,2′-dihydroxy-4-methoxybenzophenone, 3-benzophenone, (2-Hydroxy-4-methoxyphenyl)-phenylmethanone
esters of benzalmalonic acid, preferably 4-methoxybenzalmalonic acid di-2-ethylhexyl ester
triazine derivatives, such as 2,4,6-trianilino-(p-carbo-2′-ethyl-1′-hexyloxy)-1,3,5-triazine, octyl triazone and dioctyl butamido triazone
propane-1,3-diones, such as 1-(4-tert-butylphenyl)-3-(4′-methoxyphenyl)propane-1,3-dione

Possible water-soluble substances are:
2-phenylbenzimidazole-5-sulfonic acid and alkali metal, alkaline earth metal, ammonium, alkylammonium, alkanolammonium and glucammonium salts thereof
sulfonic acid derivatives of benzophenones, preferably 2-hydroxy-4-methoxybenzophenone-5-sulfonic acid and its salts
sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and salts thereof.

Typical UV-A filters are derivatives of benzoylmethane, such as 1-(4′-tert-butylphenyl)-3-(4′-methoxyphenyl)propane-1,3-dione, 4-tert-butyl-4′-methoxydibenzoylmethane, 1-(4-methoxyphenyl)-3-(4-tert-butylphenyl)propane-1,3-dione, 1-phenyl-3-(4′-isopropylphenyl)-propane-1,3-dione.

The UV-A and UV-B filters can of course also be employed in mixtures. Particularly favourable combinations comprise the derivatives of benzoylmethane, for example 4-tert-butyl-4′-methoxydibenzoylmethane and 2-cyano-3,3-phenylcinnamic acid 2-ethyl-hexyl ester (octocrylene) in combination with esters of cinnamic acid, preferably 4-methoxycinnamic acid 2-ethylhexyl ester and/or 4-methoxycinnamic acid propyl ester and/or 4-methoxycinnamic acid isoamyl ester. Such combinations are advantageously combined with water-soluble filters, such as, for example, 2-phenylbenzimidazole-5-sulfonic acid and alkali metal, alkaline earth metal, ammonium, alkylammonium, alkanolammonium and glucammonium salts thereof.

In addition to the organic substances mentioned, insoluble light protection pigments, namely finely disperse metal oxide powders, as such or in hydrophobized form, and salts are also possible for this purpose. Examples of suitable metal oxide powders or hydrophobized metal oxide powders can be titanium dioxide powder, zinc oxide powder and/or a mixed oxide powder of these with the elements Si, Ti, Al, Zn, Fe, B, Zr and/or Ce.

Typical examples are coated titanium dioxides, such as UV-Titan M212, M 262 and X 111 (Kemira), AEROXIDE® TiO2 P25, PF2, T 805 and T 817 (Evonik Corporation), Micro Titanium Dioxide MT-150 W, MT-100 AQ, MT-100 SA, MT-100 HD, MT-100 TV (Tayca), Eusolex™ T2000 (Merck), Zinc Oxide neutral H&R and Zinc Oxide NDM (Haarmann & Reimer) as well as Z-Cote and Z-Cote HP1 (BASF).

Dispersions, such as, for example, TEGO Sun TAQ 40, a 40 wt. % strength aqueous dispersion of a hydrophobized titanium dioxide (Evonik Goldschmidt) can also be used.

Typical antioxidants as part of the powdery composition according to the invention can be
amino acids like glycine, histidine, tyrosine or tryptophan and derivatives thereof,
imidazoles like urocanic acid) and derivatives thereof,
peptides, such as D,L-carnosine, D-carnosine, L-carnosine and derivatives thereof,
carotenoids, carotenes, alpha-carotene, beta-carotene, lycopene and derivatives thereof,
chlorogenic acid and derivatives thereof,
liponic acid and derivatives thereof
chelators like alpha-hydroxy-fatty acids, palmitic acid, phytic acid,
alpha-hydroxy acids like citric acid, lactic acid, malic acid
unsaturated fatty acids and derivatives thereof, like gamma-linolenic acid, linoleic acid, oleic acid,
folic acid and derivatives thereof,
ubiquinone and ubiquinol and derivatives thereof,
vitamin C and derivatives like ascorbyl palmitate, ascorbyl phosphate, ascorbyl acetate,
tocopherols and derivatives like vitamin E, vitamin E acetate,
vitamin A and derivatives like vitamin A palmitate
mannose and derivatives thereof,
superoxide dismutase.

Moisturising substance may be selected from the group consisting of ethylene glycol, butylene glycol, 2,3-butandiol, propylene glycol, dipropylene glycol, tripropylene glycol, hexylene glycol, glycerine, diglycerine, glucose, fructose, lactose, saccharose, maltose, mannitol, mannose, PEG-4 to PEG-800, like PEG-4, PEG-6, PEG-7, PEG-8, PEG-9, PEG-10, PEG-12, PEG-14, PEG-16, PEG-18, PEG-20, sorbitol, polyglyceryl sorbitol, urea, xylitol and mixtures thereof.

The deodorant and antiperspirant active compounds can be at least one selected from the group consisting of
astringent metal salts, such as aluminium hydrochlorides, aluminium hydroxylactates, aluminium zirconium hydrochlorides and zinc salts
germ-inhibiting agents, such as chitosan, phenoxyethanol, chlorhexidine gluconate or 5-chloro-2-(2,4-dichlorophenoxy)-phenol
enzyme inhibitors, such as
i) trialkyl citrates, such as trimethyl citrate, tripropyl citrate, triisopropyl citrate, tributyl citrate and triethyl citrate;
ii) sterol sulfates or phosphates, such as lanosterol, cholesterol, campesterol, stigmasterol and sitosterol sulfate and phosphate,
iii) dicarboxylic acids and esters thereof, such as, for example, glutaric acid, glutaric acid monoethyl ester, glutaric acid diethyl ester, adipic acid, adipic acid monoethyl ester, adipic acid diethyl ester, malonic acid and malonic acid diethyl ester,
iv) hydroxycarboxylic acids and esters thereof, such as, for example, citric acid, malic acid, tartaric acid or tartaric acid diethyl ester; or
v) 4-hydroxybenzoic acid and its salts and esters, N-(4-chlorophenyl)-N′-(3,4-dichlorophenyl)urea, 2,4,4′-trichloro-2′-hydroxy-diphenyl ether (Triclosan), 4-chloro-3,5-dimethyl-phenol, 2,2′-methylene-bis(6-bromo-4-chlorophenol), 3-methyl-4-(1-methylethyl)-phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide thymol, thyme oil, eugenol, clove oil, menthol, mint oil, farnesol, phenoxyethanol, glycerol monocaproate, glycerol monocaprylate, glycerol monolaurate, diglycerol monocaproate (DMC), salicylic acid N-alkylamides, such as salicylic acid n-octylamide or salicylic acid n-decylamide.
absorbers or odour maskers like benzyl acetate, p-tert-butylcyclohexyl acetate, linalyl acetate, phenylethyl acetate, linalyl benzoate, benzyl formate, allylcyclohexyl propionate, styrallyl propionate and benzyl salicylate, benzyl ethyl ether, citral, citronellal, citronellyloxyacetaldehyde, cyclamenaldehyde, hydroxycitronellal, jonones, methyl cedryl ketone, anethole, citronellol, eugenol, isoeugenol, geraniol, linalool, phenylethyl alcohol, terpineol or terpenes.

The biogenic substances may be selected from at least one of the group consisting of tocopherol, tocopherol acetate, tocopherol palmitate, ascorbic acid, (deoxy)ribonucleic acid and fragmentation products thereof, beta-glucans, retinol, bisabolol, allantoin, phytantriol, panthenol, pantothenic acid, fruit acids, alpha-hydroxy acids, amino acids, ceramides, pseudoceramides, essential oils, plant extracts, such as, for example, prunus extract, bambara nut extract and vitamin complexes.

The insect repellent active compounds may be selected from the group consisting of N,N-diethyl-m-toluamide, 1,2-pentanediol, 3-(N-n-butyl-N-acetyl-amino)-propionic acid ethyl ester and butyl acetylaminopropionate.

Examples of bleaching agents suitable for the present invention are hydroquinone, zinc peroxide, urea peroxide, hydrogen peroxide and/or organic peroxides.

In a special embodiment of the invention the core of the core-shell particles is water, and the shell consists of hydrophobized fumed silica particles that are obtained by reacting a hydrophilic fumed silica having a BET surface area of from 30 to 500 m²/g.

Furthermore in an advantageous embodiment of the invention the hydrophobized fumed silica particles are obtained by reacting a hydrophilic fumed silica having a BET surface area from 270 to 330 m²/g with hexamethyldisilazane to give hydrophobized fumed silica particles having a BET surface area of from 200 to 290 m²/g and a carbon content of 2 to 4 wt.-% and methanol wettability of at least 50.

The special embodiment may further comprise as carrier a precipitated silica having a BET surface area from 400 to 500 m²/g, a particle size d50, determined by laser diffraction, from 4 to 6 μm and a DBP (dibutyl phthalate) absorption, in g DBP/100 g silica, from 300 to 350.

The special embodiment may also further comprise as carrier a precipitated silica having a BET surface area from 150 to 600 m²/g, a particle size d50, determined by laser diffraction, from 4 to 600 μm and a DBP (dibutyl phthalate) absorption, in g DBP/100 g silica, from 250 to 350.

The special embodiment may further comprise as carrier a precipitated silica having a BET surface area from 400 to 600 m²/g, a particle size d50, determined by laser diffraction, from 9 to 12 μm and a DBP (dibutyl phthalate) absorption, in g DBP/100 g silica, from 250 to 290.

The special embodiment may further comprise as carrier a precipitated silica having a BET surface area from 160 to 200 m²/g, a particle size d50, determined by laser diffraction, from 7 to 11 μm and a DBP (dibutyl phthalate) absorption, in g DBP/100 g silica, from 220 to 250.

In addition the special embodiments may further comprise as at least partially water soluble liquid and/or water reactive substrate, each located in and/or on the carrier, and texturizers, preservatives, pigments, UV light protection filters, antioxidants, moisturizing substances, deodorant, antiperspirant active compounds, biogenic substances, insect repellent active compounds, bleaching agents antioxidant, bleach, antibacterial substances, antifungal substances, flavors, fragrances, acids, alkalis, enzymes or mixtures thereof.

Another object of the invention is a process for the manufacture of a liquid or creamy composition, starting from the powdery composition according to the invention. This powdery composition is activated by an energy input in form of pressure, shear, temperature, vibration and/or solvent addition, the energy input being sufficient to release the liquid water mixture or the liquid aqueous phase from the core-shell material, the liquid water mixture or the liquid aqueous phase subsequently is
a) dissolving or flushing the at least partially water soluble liquid from the carrier and/or
b) activating the water reactive substrate in and/or on the carrier and subsequently dissolving or flushing the product or products obtained by activating from the carrier resulting in a liquid or creamy composition.

“Creamy” is understood to mean a range from more liquid compositions to more gel like compositions to creamy compositions.

The powdery composition may be applied or delivered to the desired surface or object through various delivery means and devices prior to or after activation by an energy input. In one embodiment, the powdery composition may be stored in a container suitably sized to receive various sized and shaped applicator tools capable of collecting a volume of the powdery composition from the container without activating the powdery composition such as, but not limited to, brushes, swabs, sponges, and fingers until at such time that the powdery composition is applied by the applicator tool to the desired surface. In some embodiments, the applicator tools may preferably contain fibers or similar materials in which the powdery composition may temporarily adhere to without being activated. In some instances, the powdery composition has been applied to the desire surface using the applicator tools without activating the powdery composition; the powdery composition may then be activated by applying a suitable energy input, for example by shear force from applying finger pressure to convert the powdery composition to a creamy or liquid composition. In other instances, the powdery composition is activated as the applicator tool applies the powdery composition to the desired surface. Examples of such applicator tools may include but are not limited to shaker containers, brushes such as paintbrushes, cosmetic and make-up brushes, paint rollers, brooms, mops, sponges, cotton balls, cotton swabs, and fingers. In some instances, the powdery compositions are suitable for cosmetic preparations, such as powders, in various products including, but not limited to, moisturizers, lotions, mascara, lipstick, lip gloss, sunscreen and fragrances. In other instances, the powdery compositions are suitable for industrial and commercial applications including in paint and stain products and formulations, and other surface treatment products. The type of container and applicator tool would depend on the specific use of such powdery composition.

In one embodiment, the powdery composition may be stored in a container combined with a spray apparatus and nozzle. In this embodiment, the powdery composition travels through the spray apparatus and nozzle without being activated and delivered to the desired surface. Once the powdery composition has been deposited onto its desired surface, it can be activated by applying an adequate energy input. Examples of such spray apparatus and nozzle may include but are not limited to hand pump spray dispensers, pressurized spray dispensers, and electric spray dispensers.

In another embodiment, the powdery composition may be delivered to the desired surface after activation by an adequate energy input. In one embodiment, the powdery composition may be stored in a container combined with a spray apparatus and nozzle. In this embodiment, as the powdery composition travels through the spray apparatus and nozzle, sufficient energy input has been applied such that the liquid water mixture or the liquid aqueous phase from the core-shell material of the powdery composition has been released as it exits the spray nozzle and delivered to the desired surface. Examples of such spray apparatus and nozzle may include but are not limited to hand pump spray dispensers, pressurized spray dispensers, and electric spray dispensers.

Having generally described this invention, a further understanding can be obtained by reference to certain specific examples which are provided for illustration only, and are not intended to be limiting.

### Examples

### Example 1: Powder-to-Cream Matte Lip Product Formulation According to the Invention

To make a novel Powder-to-Cream Matte Lip Product formulation according to the invention, a solution (i.e., emollient/pigment solution) of PEG/Dimethicone/pigment/DI water dilution was created by mixing 35.2 wt.-% Carbowax® PEG, 400 (The DOW Chemical Company), 24.2 wt.-% DIMETHISIL® DM-20 (20 cPs) (Chemsil Silicones, Inc.), 39.7 wt.-% deionized (DI) water, and 0.5 wt.-% Unicert Red K7057-J Red 33 (Sensient Cosmetic Technologies), and 0.4 wt.-% personal care approved preservative. This solution was then added to SIPERNAT® 500LS silica (i.e., carrier) from Evonik Corporation in 3-4 addition steps using low shear mixing by hand using a metal spatula. Use low shear mixing until the solution has completely absorbed into the silica and has turned into dry powder. This mixing of the solution and silica resulted in a mix that consisted of 70 wt.-% emollient/pigment solution, the liquid, and 30 wt-%, the carrier. This powder product will now be referred to as carrier powder.

The carrier powder was further combined with additional materials resulting in a mix that consisted of 2.0 wt-% AEROSIL® R812S from Evonik Corporation, 10.0 wt-% Spheron N-2000 J (Presperse Corporation), and 10.0 wt-% Cloisonne Blue Flambe and 78.0 wt-% carrier powder.

The dry liquid mixture was produced by adding into a blender 10.0 wt.-% of AEROSIL® R812S (Evonik Corporation), 0.85 wt.-% Unicert Red K7057-J (Sensient Cosmetic Technologies), 26.55 wt.-% DI water, and 62.0 wt.-% glycerin and 0.6 wt.-% personal care approved preservative. The lid was placed tightly on the blender and the dry liquid mixture was blended at the highest setting (Liquify) at high speed for one minute. During the one minute of mixing, the entire blender was tilted at a 50-60 degree angle from left to right with about 10 second hold times.

Twenty-five percent by weight of the liquid/carrier was then gently mixed (tumbled, tossed or folded in) with 75 wt. % of dry liquid mixture to make the final powder-to-cream matte lip product.

This powder-to-cream matte lip product was then stored in a plastic container. The powdery composition was able to be created, and stay, as a powder.

### Example 2: Powder-to-Cream Glossy Lip Product Formulation According to the Invention

To make a novel Powder-to-Cream Glossy Lip Product formulation according to the invention, a solution (i.e., emollient/pigment solution) PEG/Dimethicone/pigment/DI water dilution was created by mixing 35.0 wt.-% Carbowax® PEG, 400 (The DOW Chemical Company), 24.2 wt.-% Dimethisil® DM-20 (20 cPs) (Chemsil Silicones, Inc.), 39.7 wt.-% deionized (DI) water, and 0.7 wt.-% Unicert Red K7057-J Red 33 (Sensient Cosmetic Technologies), and 0.4 wt.-% personal care approved preservative. This solution was then added to SIPERNAT® 500LS silica (i.e., carrier) from Evonik Corporation in 3-4 addition steps using low shear mixing by hand using a metal spatula. Use low shear mixing until the solution has completely absorbed into the silica and has turned into dry powder. This mixing of the solution and silica resulted in a mix that consisted of 70 wt.-% emollient/pigment solution, the liquid, and 30 wt-%, the carrier. This powder product will now be referred to as carrier powder.

The carrier powder was further combined with additional materials resulting in a mix that consisted of 2.0 wt-% AEROSIL® R812S from Evonik Corporation, 10.0 wt-% Spheron N-2000 J (Presperse Corporation), and 10.0 wt-% Cloisonne Blue Flambe and 78.0 wt.-% carrier powder.

The dry liquid mixture was produced by adding into a blender 10.0 wt.-% of AEROSIL® R812S (Evonik Corporation), 0.85 wt.-% Unicert Red K7057-J (Sensient Cosmetic Technologies), 3.6 wt.-% DI water, and 84.95 wt.-% glycerin and 0.6 wt.-% personal care approved preservative. The lid was placed tightly on the blender and the mixture was blended at the highest setting (Liquify) at high speed for one minute. During the one minute of mixing, the entire blender was tilted at a 50-60 degree angle from left to right with about 10 second hold times. Twenty-five percent by weight of the liquid/carrier was then gently mixed (tumbled, tossed or folded in) with 75 wt. % of dry liquid mixture to make the final powder-to-cream glossy lip product.

This powder-to-cream glossy lip product was then stored in a plastic container. The powdery composition was able to be created, and stay, as a powder.

TAB. 1
Ingredient of the formulations of Examples 1 to 2
(wt.-% of the total powdery composition)

| | Ex. 1 | Ex. 2 |
|---|---|---|
| Dimethicone | 3.3 | 3.3 |
| Pigments | 3.21 | 3.21 |
| Glycerin | 46.5 | 63.72 |
| Preservative | 0.5 | 0.5 |
| Spheron N-2000J | 2.5 | 2.5 |
| Carbowax 400 NF | 4.8 | 4.8 |
| Deionized Water | 25.34 | 8.12 |
| AEROSIL ® R 812S | 8 | 8 |
| SIPERNAT ® 500 LS | 5.85 1 | 5.85 |

1. A powdery composition, comprising:
a) core-shell particles having
a shell of hydrophobized particles; and
a core comprising:
water; and
10 to 95 wt.-% of a total core weight of a dissolved or emulsified material; and
b) a carrier powder comprising:
a carrier; and
at least one of an at least partially water soluble liquid and/or a water reactive substrate reversibly bound to the carrier by at least one of capillary action, absorption and adsorption;
the carrier comprises at least one material selected from the group consisting of silica, a silica-metal mixed oxide, fumed silica, phyllosilicate, clay, diatomaceous earth, starch, hollow glass spheres, nylon, sugar, cyclodextrines and polysaccharides, and
wherein a weight ratio of the carrier powder to the core-shell particles is from 1:1 to 1:9,
wherein
the shell particles comprise hydrophobized silica particles,
wherein at least one of
a) the core of the core-shell particles
and
b) the at least partially water soluble liquid and water reactive substrate comprises at least one material selected from the group consisting of a texturizer, preservative, pigment, exfoliant, UV light protection filter, an antioxidant, a moisturizing substance, a deodorant, an antiperspirant active compound, a biogenic substance, an insect repellent active compound, a bleaching agent, an antioxidant, a bleach, an antibacterial substance, an antifungal substance, a flavor, a fragrance, an acid, an alkali, and an enzyme.

2. The powdery composition according to claim 1, wherein the hydrophobized silica particles have a BET surface area from 200 to 290 m²/g.

3. The powdery composition according claim 2, wherein the hydrophobized silica particles have a carbon content from 2 to 4 wt.-%.

4. The powdery composition according claim 3, wherein the hydrophobized silica particles have a methanol wettability of at least 50.

5. The powdery composition according claim 4, wherein the hydrophobized silica particles are obtained by reacting a hydrophilic fumed silica having a BET surface area of from 270 to 330 m²/g with hexamethyldisilazane.

6. The powdery composition according to claim 1, wherein the hydrophobized silica particles are aggregated fumed silica particles.

7. The powdery composition according to claim 1, wherein a dissolved or emulsified material content of the powdery composition is 30 to 70 wt.-% of the total powdery composition weight.

8. The powdery composition according to claim 1, wherein a dissolved or emulsified material content of the powdery composition is 40 to 50 wt.-% of the total powdery composition weight.

9. The powdery composition according to claim 1, wherein the dissolved or emulsified material content of the powdery composition is 46 wt.-% of the total powdery composition weight.

10. The powdery composition according to claim 1, wherein the dissolved or emulsified material content of the powdery composition is 47 wt.-% of the total powdery composition weight.

11. The powdery composition according to claim 1, wherein the dissolved or emulsified material comprises at least one material selected from the group consisting of ethylene glycol, butylene glycol, 2,3-butandiol, propylene glycol, dipropylene glycol, tripropylene glycol, hexylene glycol, glycerine, diglycerine, glucose, fructose, lactose, saccharose, maltose, mannitol, mannose, PEG-4 to PEG-800, like PEG-4, PEG-6, PEG-7, PEG-8, PEG-9, PEG-10, PEG-12, PEG-14, PEG-16, PEG-18, PEG-20, sorbitol, polyglyceryl sorbitol, urea, xylitol and mixtures thereof.

12. The powdery composition according to claim 7, wherein the dissolved or emulsified material is glycerine.

13. The powdery composition according to claim 1, wherein the water reactive substrate comprises at least one of Argon Oil, Butylene Glycol, C12-C15 Alkyl Benzoate, Caprylic/Capric Triglyceride, Castor, Cyclopentasiloxane, Decamethylcyclopentasiloxane, Ethylhexyl Palmitate/Tegosoft OP, Glycerin, Grapeseed, Hexadecanoic Acid, 2-ethylhexyl ester Isocetyl Palmitate, Jojoba, Macadamia, Mineral Oil, Olive, Paraffin Oil, PEG 400, Polydimethylsiloxane, PPG-15 Stearyl/Ether, Propylene Glycol, Rapeseed, Silicone 350, Silicone Fluid 50, Soybean, Soybean Oil, and Sunflower Oil.

14. The powdery composition according to claim 1, further comprising a free-flow agent.

15. The powdery composition according to claim 1, wherein an average particle size of the core-shell particles is from 2 to 20 um.

16. The powdery composition according to claim 1, wherein the texturizer is selected from the group consisting of carbomer, polymers, inorganic powders such as talc, kaolin, mica, sericite, dolomite, phlogopite, synthetic mica, lepidolite, biotite, lithia mica, vermiculite, magnesium carbonate, calcium carbonate, aluminum silicate, barium silicate, calcium silicate, magnesium silicate, strontium silicate, tungstenic acid metal salts, magnesium, silica, zeolite, barium sulfate, calcined calcium sulfate, calcium phosphate, fluorapatite, hydroxyapatite, titania, fumed titania, alumina and fumed alumina.

17. The powdery composition according to claim 1, wherein the preservative is selected from the group comprising at least one of methylparaben, ethylparaben, butylparaben, propylparaben, and PHENONIP.

18. The powdery composition according to claim 1, wherein the UV is selected from the group comprising at least one of (2-Hydroxy-4-methoxyphenyl)-phenylmethanone, 1-(4-methoxyphenyl)-3-(4-tert-butylphenyl)propane-1,3-dione, titanium dioxide powder and zinc oxide powder.

19. The powdery composition according to claim 1, wherein the weight ratio of the carrier powder to the core-shell particles is 1:3.

20. The powdery composition according to claim 1, wherein the carrier is a precipitated silica,
a BET surface area of the precipitated silica is from 400 to 500 m²/g,
a particle size d50 of the precipitated silica, determined by laser diffraction, is from 4 to 600 μm, and
a DBP (dibutyl phthalate) absorption of the precipitated silica, in g DBP/100 g silica, is from 300 to 350.

21. A method of using the powdery composition according to claim 1 comprising:
a) applying the powdery composition to a desired surface;
b) activating the powdery composition by applying an energy input to the powdery composition sufficient to release the water and dissolved or emulsified material from the core-shell.

22. The method according to claim 21, where the energy input is provided in the form of pressure, shear, temperature, vibration, solvent addition or combination thereof.

23. A method of using the powdery composition according to claim 1 in a formulation for a moisturizer, lotion, mascara, lipstick, lip gloss, sunscreen and fragrance.

24. A powdery composition, comprising:
a) core-shell particles having
a shell of hydrophobized particles; and
a core comprising:
water; and
10 to 95 wt.-% of a total core weight of a dissolved or emulsified material; and
b) a carrier powder comprising:
a carrier; and
at least one of an at least partially water soluble liquid and/or a water reactive substrate reversibly bound to the carrier by at least one of capillary action, absorption and adsorption;
wherein a weight ratio of the carrier powder to the core-shell particles is from 1:1 to 1:9,
wherein
the shell particles comprise hydrophobized silica particles,
a BET surface area of the hydrophobized silica particles is from 200 to 290 m²/g,
a carbon content of the hydrophobized silica particles is 2 to 4 wt.-%,
a methanol wettability of the hydrophobized silica particles is at least 50,
the hydrophobized silica particles are obtained by reacting a hydrophilic fumed silica having a BET surface area of from 270 to 330 m²/g with hexamethyldisilazane,
wherein at least one of
a) the core of the core-shell particles
and
b) the at least partially water soluble liquid and water reactive substrate comprises at least one material selected from the group consisting of a texturizer, preservative, pigment, exfoliant, UV light protection filter, an antioxidant, a moisturizing substance, a deodorant, an antiperspirant active compound, a biogenic substance, an insect repellent active compound, a bleaching agent, an antioxidant, a bleach, an antibacterial substance, an antifungal substance, a flavor, a fragrance, an acid, an alkali, and an enzyme, and
wherein
the carrier is a precipitated silica,
a BET surface area of the precipitated silica is from 50 to 1000 m²/g,
a particle size d50 of the precipitated silica, determined by laser diffraction, is from 4 to 50 μm, and
a DBP (dibutyl phthalate) absorption of the precipitated silica, in g DBP/100 g silica, is from 200 to 400.

25. A powdery composition, comprising:
a) core-shell particles having
a shell of hydrophobized particles; and
a core comprising:
water; and
10 to 95 wt.-% of a total core weight of a dissolved or emulsified material; and
b) a carrier powder comprising:
a carrier; and
at least one of an at least partially water soluble liquid and/or a water reactive substrate reversibly bound to the carrier by at least one of capillary action, absorption and adsorption;
wherein a weight ratio of the carrier powder to the core-shell particles is from 1:1 to 1:9,
wherein
the shell particles comprise hydrophobized silica particles,
a BET surface area of the hydrophobized silica particles is from 200 to 290 m²/g,
a carbon content of the hydrophobized silica particles is 2 to 4 wt.-%,
a methanol wettability of the hydrophobized silica particles is at least 50,
the hydrophobized silica particles are obtained by reacting a hydrophilic fumed silica having a BET surface area of from 270 to 330 m²/g with hexamethyldisilazane,
wherein
the carrier is a fumed silica,
a BET surface area of the fumed silica is from 100 to 350 m²/g,
wherein at least one of
a) the core of the core-shell particles
and
b) the at least partially water soluble liquid and water reactive substrate comprises at least one material selected from the group consisting of a texturizer, preservative, pigment, exfoliant, UV light protection filter, an antioxidant, a moisturizing substance, a deodorant, an antiperspirant active compound, a biogenic substance, an insect repellent active compound, a bleaching agent, an antioxidant, a bleach, an antibacterial substance, an antifungal substance, a flavor, a fragrance, an acid, an alkali, and an enzyme.
